(19) United States
(12) Patent Application Publication
(10) Pub. No.: US 2023/0108332 A1
(43) Pub. Date: Apr. 6, 2023

(54) CATHETER

(71) Applicant: ASAHI INTECC CO., LTD., Seto-shi (JP)

(72) Inventor: Nawaporn HARNMONTRI, Seto-shi (JP)

(73) Assignee: ASAHI INTECC CO., LTD., Seto-shi (JP)

(21) Appl. No.: 18/076,719

(22) Filed: Dec. 7, 2022

Related U.S. Application Data
(63) Continuation of application No. PCT/JP2020/026490, filed on Jul. 6, 2020.

(51) Int. Cl. A61B 17/3207; A61M 25/00

(52) U.S. Cl. CPC A61B 17/3207; A61M 25/0068

(57) ABSTRACT

A catheter includes a hollow shaft and a metal distal tip connected to a distal end of the shaft. The distal tip includes, between a distal end of the distal tip and the distal end of the shaft, an enlarged diameter portion where an outer diameter in a direction perpendicular to an axial direction of the shaft is largest, and an outer diameter at the enlarged diameter portion is larger than an outer diameter of the distal end of the shaft. An outer shape of the distal tip sandwiching the enlarged diameter portion between a distal and a proximal end side may be formed as a curved surface with gradually changing inclination. The distal tip may have an outer shape between the enlarged diameter portion and the distal end of the shaft that is outwardly concave.

### CROSS REFERENCE TO RELATED APPLICATION

This application is a continuation application of International Application No. PCT/JP2020/026490, filed Jul. 6, 2020, the entire disclosure of which is incorporated herein by reference.

### TECHNICAL FIELD

The present disclosure relates to a catheter.

### BACKGROUND

Catheters for improving the blood flow by removing occlusions that block blood vessels, such as a chronic total occlusion (CTO), are known.

Among such catheters, there is known a catheter provided with a metal distal tip at a distal end for passing the catheter through a hard lesion site (see, for example, Japanese Unexamined Patent Application Publication No. 2018-519957).

### SUMMARY

It is required for catheters intended to be passed through a hard lesion that the catheter is easily passed through a lesion site and the damage to the catheter can be prevented when the catheter is passed through the lesion site.

The present disclosure has been made based on the above requirements, and an object of the present disclosure is to provide a catheter suitable for being passed through a hard site.

To achieve such an object, a catheter according to an aspect includes a hollow shaft and a distal tip formed of metal and connected to a distal end of the hollow shaft. The distal tip includes, between a distal end of the distal tip and the distal end of the hollow shaft, an enlarged diameter portion where an outer diameter in a direction perpendicular to an axial direction of the hollow shaft is largest. The outer diameter of the enlarged diameter portion is larger than an outer diameter of the distal end of the hollow shaft.

In the catheter described above, the distal tip may be formed an outer shape between a distal end side and a proximal end side sandwiching the enlarged diameter portion by a curved surface having an inclination that gradually changes. In the catheter described above, the distal tip may be formed so that an outer shape between the enlarged diameter portion and the distal end of the hollow shaft is outwardly concave. Further, a slit may be formed in an outer peripheral surface of the distal tip on a distal end side of the enlarged diameter portion. Moreover, a protruding portion extending spirally and protruding radially outward may be formed on the outer peripheral surface of the distal tip.

### BRIEF DESCRIPTION OF THE DRAWINGS

FIG. 1 is a diagram of a configuration of a catheter according to a first embodiment.

FIG. 2 is a diagram of a configuration of the vicinity of a distal end of the catheter illustrated in FIG. 1.

FIG. 3 is a diagram of a configuration of the vicinity of the distal end of a catheter according to a second embodiment.

FIG. 4 is a diagram of a configuration of the vicinity of the distal end of a catheter according to a third embodiment.

FIGS. 5A and 5B are diagrams of a configuration of a distal tip of a catheter according to a fourth embodiment.

FIGS. 6A and 6B are diagrams of a configuration of a distal tip of a catheter according to a fifth embodiment.

### DETAILED DESCRIPTION

Catheters according to several embodiments will be described with reference to the drawings, but the present invention is not limited only to the embodiments described in the drawings.

As used herein, the terms “distal end side” and “distal end direction” refer to a side and a direction where a distal tip is positioned with respect to a hollow shaft in a direction along a longitudinal direction of a catheter (direction along an axial direction of the hollow shaft). The terms “proximal end side” and “proximal end direction” refer to a side and a direction that are opposite to the distal end side and the distal end direction at a side and in a direction along a direction along the longitudinal direction of the catheter. Further, the term “distal end” refers to an end portion at the distal end side in any member or site, and the term “proximal end” refers to an end portion at the proximal end side in any member or site, respectively.

### First Embodiment

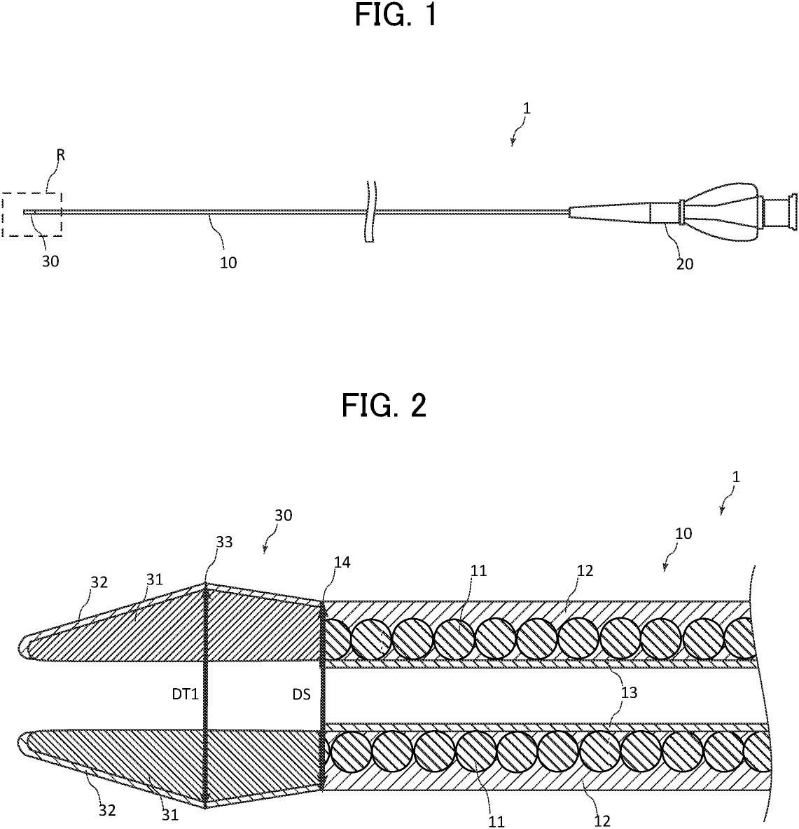

FIG. 1 is a diagram of a configuration of a catheter according to a first embodiment. A catheter 1 includes a shaft 10, a connector 20 provided on the proximal end side of the shaft 10, and a distal tip 30 connected to the distal end side of the shaft 10.

FIG. 2 illustrates a configuration of the vicinity of a distal end of the catheter illustrated in FIG. 1, and more specifically, is an enlarged view of a cross section of a region R. The shaft 10 is a hollow member and includes a coil body 11, an outer layer 12, and an inner layer 13.

The coil body 11 is an example of a reinforcing body that reinforces the shaft 10, and is formed by spirally winding a metal wire. The coil body 11 may be formed by spirally winding one wire (solid wire coil), or may be formed by spirally winding a plurality of wires (stranded coil). Considering a rotation force of the catheter 1, the coil body 11 is preferably a stranded coil. The metal material of the wire of the coil body 11 may be stainless steel (SUS304, SUS316, and the like), gold, platinum, tungsten, nickel, alloys containing these elements, and the like.

The outer layer 12 is a layer formed of a resin, for example, and is provided to cover an outer periphery of the coil body 11. The resin material forming the outer layer 12 is not particularly limited, and may be, for example, a polyamide, a polyamide elastomer, a polyester, polyurethane, and the like.

The inner layer 13 is a layer formed of a resin, for example, and is provided to cover an inner periphery of the coil body 11. The resin material forming the inner layer 13 is not particularly limited, but in consideration of slidability between the shaft 10 and an instrument (such as a guide wire) to be inserted inside the shaft 10, PTFE (polytetrafluoroethylene) is preferable.

The distal tip 30 that is hollow and made of a metal, is connected to a distal end portion 14 of the shaft 10. The distal tip 30 includes a main body portion 31 and a coating layer 32.

The main body portion 31 is formed of a metal material. The metal material forming the main body portion 31 is not particularly limited, and may be stainless steel (SUS304, SUS316, and the like), gold, platinum, tungsten, platinum, nickel, alloys containing these elements, and the like. The distal tip 30 may be formed of a radiopaque metal material so that it is possible to grasp a position of a distal end of the catheter 1 in a radioscopic image.

The coating layer 32 is a layer formed of a resin, for example, and is provided to cover an outer periphery of the main body portion 31. The resin material forming the coating layer 32 is not particularly limited, and may be, for example, a polyamide, a polyamide elastomer, a polyester, polyurethane, and the like.

The distal tip 30 includes, between the distal end of the distal tip 30 and the distal end portion 14 of the shaft 10, an enlarged diameter portion 33 where an outer diameter in a direction perpendicular to the axial direction (longitudinal direction) of the shaft 10 is largest. An outer diameter DT1 of the enlarged diameter portion 33 is larger than an outer diameter DS of the distal end portion 14 of the shaft 10. An outer diameter of the main body portion 31 in the enlarged diameter portion 33 is also larger than the outer diameter DS of the distal end portion 14 of the shaft 10.

The distal tip 30 is formed to have a tapered shape in which the outer diameter decreases from the enlarged diameter portion 33 toward the distal end, and the outer diameter decreases from the enlarged diameter portion 33 toward the distal end portion 14 of the shaft 10. A rear end of the distal tip 30 has the same outer diameter as an outer diameter DS, and an outer peripheral edge of the rear end of the distal tip 30 is connected to an outer peripheral edge of the distal end portion 14 of the shaft 10. The shaft 10 and the distal tip 30 are connected to have a continuous outer periphery.

The distal tip 30 and the shaft 10 are formed so that a rear end surface of the distal tip 30 and a distal end surface of the shaft 10 contact, at least in part, each other, and the rear end surface of the distal tip 30 and the distal end surface of the shaft 10 are joined to each other. As illustrated in FIG. 2, the coil body 11 and the main body portion 31 may be joined by welding at a surface where the metal materials contact each other. Moreover, the coating layer 32 and the outer layer 12 may be joined by thermal welding at a site where the resins contact each other.

If the catheter 1 is advanced through a hard lesion (for example, a calcified lesion), the shaft 10 passes through a portion through which the enlarged diameter portion 33 of the distal tip 30 passes. In the catheter 1, the outer diameter of the distal end portion 14 of the shaft 10 is smaller than the outer diameter of the enlarged diameter portion 33, so that a contact area between the lesion site and the shaft 10 is reduced. By reducing the contact area, it is possible to appropriately reduce the probability that the shaft 10 gets caught in the lesion or is damaged by the lesion. In addition, by reducing the contact area, it is possible to reduce the contact resistance by the shaft 10 when advancing the catheter 1, and to improve the pushing force (pushability) of the catheter 1.

### Second Embodiment

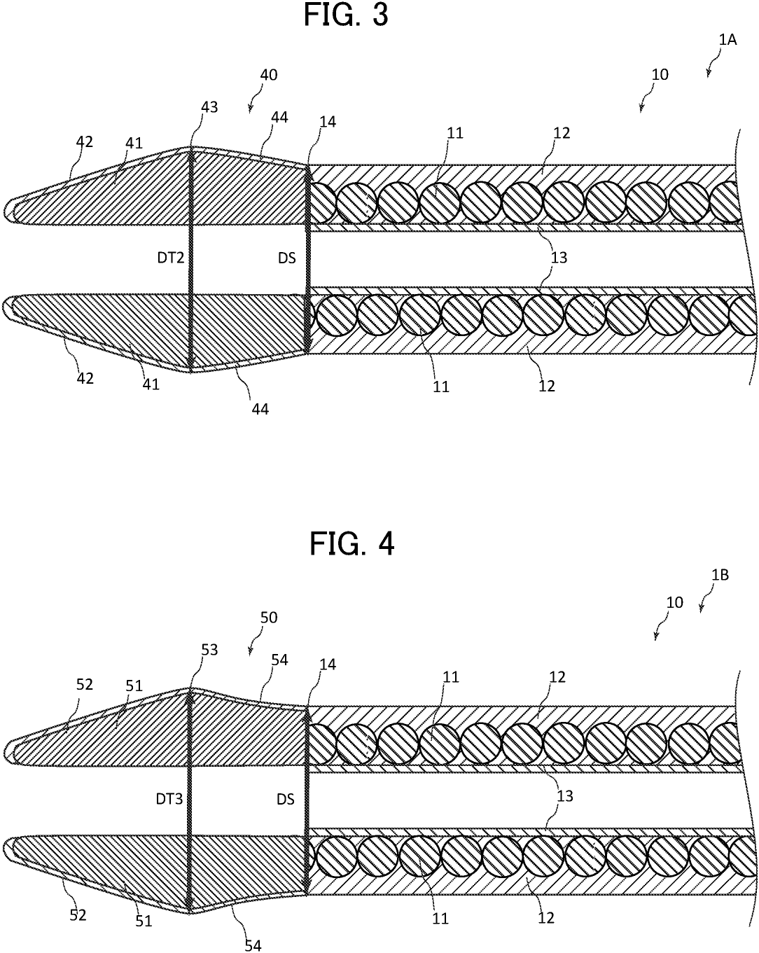

FIG. 3 is a diagram of a configuration of the vicinity of a distal end of a catheter according to a second embodiment, and illustrates a cross-section of the distal end of the catheter. A catheter 1A according to the second embodiment differs from the catheter 1 in the shape of the distal tip. Portions similar to those of the above-described embodiment are given the same reference symbols, and duplicated description is omitted. A distal tip 40 according to the second embodiment includes a main body portion 41 and a coating layer 42. The main body portion 41 and the coating layer 42 are similar to the main body portion 31 and the coating layer 32, except that the shapes are different.

The distal tip 40 includes, between the distal end and the distal end portion 14 of the shaft 10, an enlarged diameter portion 43 where the outer diameter is largest. An outer diameter DT2 at the enlarged diameter portion 43 is larger than the outer diameter DS of the distal end portion 14 of the shaft 10. The outer diameter of the main body portion 41 at the enlarged diameter portion 43 is also larger than the outer diameter DS of the distal end portion 14 of the shaft 10. The distal tip 40 includes an outer peripheral surface 44 extending from the distal end side of the enlarged diameter portion 43 to the distal end portion 14 of the shaft 10 via the enlarged diameter portion 43. As illustrated in FIG. 3, the outer peripheral surface 44 is formed with an outwardly convex curved surface having an inclination gradually changing in a longitudinal sectional view of the catheter 1A, that is, a gently curved surface. The outwardly convex curved surface is convex with respect to the radial direction (i.e., it is radially convex). Here, “inclination” refers to an amount of change of the outer peripheral surface 44 in a radial direction of the catheter 1A, with respect to the length in the axial direction of the catheter 1A, in a longitudinal sectional view.

In other words, the distal tip has an outer shape formed between a distal end side and a proximal end side of the distal tip, and the outer shape is formed by a curved surface having an inclination gradually changing in a longitudinal sectional view. As shown in FIG. 3, the distal and proximal end sides sandwich the enlarged diameter portion.

According to the catheter 1A according to the present embodiment, in addition to the effects of the first embodiment, when the catheter 1A is advanced toward the distal end side, it is possible to appropriately prevent damage to normal blood vessels that contact the enlarged diameter portion 43 of the distal tip 40 and the outer peripheral surface of the catheter 1A on the proximal end side of the enlarged diameter portion 43.

### Third Embodiment

FIG. 4 is a diagram of a configuration of the vicinity of a distal end of a catheter according to a third embodiment, and illustrates a cross-section of the distal end of the catheter illustrated in FIG. 2. A catheter 1B according to the third embodiment differs from the catheter 1 in the shape of the distal tip. Portions similar to those of the above-described embodiment are given the same reference symbols, and duplicated description is omitted. A distal tip 50 according to the third embodiment includes a main body portion 51 and a coating layer 52. The main body portion 51 and the coating layer 52 are similar to the main body portion 31 and the coating layer 32, except that the shapes are different.

The distal tip 50 includes, between the distal end and the distal end portion 14 of the shaft 10, an enlarged diameter portion 53 where the outer diameter is largest. An outer diameter DT3 at the enlarged diameter portion 53 is larger than the outer diameter DS of the distal end portion 14 of the shaft 10. The outer diameter of the main body portion 51 at the enlarged diameter portion 53 is also larger than the outer diameter DS of the distal end portion 14 of the shaft 10. In the distal tip 50, an outer peripheral surface 54 extends from the distal end side of the enlarged diameter portion 53 to the distal end portion 14 of the shaft 10 and is formed with a more outwardly concave curved surface at the distal end portion 14 of the shaft 10 than at the enlarged diameter portion 53. The outwardly concave curved surface is concave with respect to the radial direction (i.e., it is radially concave).

If the catheter 1B is advanced through a hard lesion (for example, a calcified lesion), in a portion through which the enlarged diameter portion 53 of the distal tip 50 passes, it is possible to more effectively reduce the contact of the outer peripheral surface 54 at the proximal end side of the enlarged diameter portion 53 with the lesion. Thus, it is possible to appropriately reduce the probability that the outer peripheral surface 54 gets caught in the lesion. It is possible to reduce the probability that the enlarged diameter portion 53 of the distal tip 50 contacts the lesion, and thus, it is possible to reduce the contact resistance when advancing the catheter 1B, and to improve the pushing force (pushability) of the catheter 1B. If the catheter 1B is advanced toward the distal end side, the probability that the outer peripheral surface 54 of the distal tip 50 contacts a normal blood vessel can be reduced, and it is possible to appropriately prevent damage to blood vessels.

### Fourth Embodiment

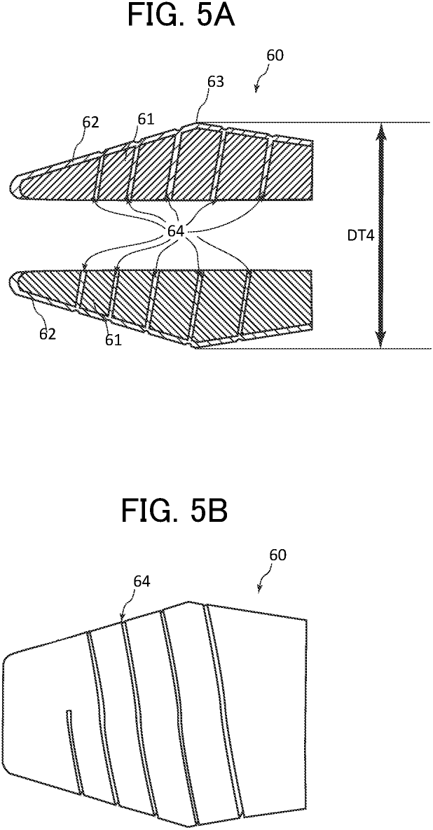

FIGS. 5A and 5B are diagrams of a configuration of a distal tip of a catheter according to a fourth embodiment, in which FIG. 5A is a cross-sectional view of the distal tip and FIG. 5B is a side view of the distal tip. In contrast to the distal tip 30 according to the first embodiment, a distal tip 60 according to the fourth embodiment is formed with a slit 64. The distal tip 60 includes a main body portion 61 and a coating layer 62. The main body portion 61 is similar to the main body portion 31, except that the main body portion 61 is formed with the slit 64. The coating layer 62 is similar to the coating layer 32, except that the coating layer 62 penetrates into the slit 64.

In the main body portion 61, as illustrated in FIG. 5B, the slit 64 is formed in a spiral shape. The slit 64 is provided to improve the flexibility of the main body portion 61 by cutting into the main body portion 61. In the present embodiment, the slit 64 is formed in a spiral shape, and thus, the distal tip 60 can be bent equally easily in any direction. Since a pitch of the slit 64 is formed to decrease toward the distal end, a distal end region of the distal tip 60 is particularly flexible. Therefore, it is possible to reduce the risk of perforating a blood vessel with the distal tip 60.

As illustrated in FIG. 5A, the distal tip 60 is configured such that the resin of the coating layer 62 penetrates inside the slit 64 of the main body portion 61.

The distal tip 60 is provided with the slit 64, so that the distal tip 60 can be easily bent while maintaining the hardness of the distal tip 60. The outer peripheral surface of the distal tip 60 is covered with the coating layer 62, and thus, it is possible to smooth the surface of the distal tip 60. As a result, the catheter provided with the distal tip 60 can be easily passed through a hard lesion and exhibits good followability even in a tortuous blood vessel such as a peripheral blood vessel.

In the distal tip 60, the resin of the coating layer 62 penetrates inside the slit 64, so that it is possible to firmly fix the coating layer 62 to the main body portion 61. Therefore, it is possible to appropriately prevent the coating layer 62 from detaching from the main body portion 61 due to friction with the outside. Thus, the smoothness of the outer peripheral surface of the distal tip 60 can be maintained, even if the distal tip 60 is passed through a hard lesion.

### Fifth Embodiment

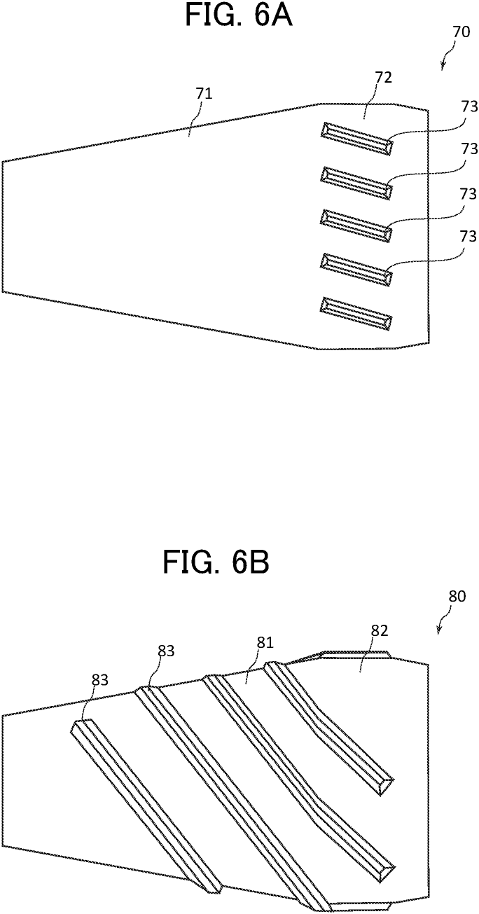

FIGS. 6A and 6B are diagrams of a configuration of a distal tip of a catheter according to a fifth embodiment, in which FIG. 6A is a side view of the distal tip and FIG. 6B is a side view of a distal tip according to a modification modified from the configuration illustrated in FIG. 6A.

As shown in FIG. 6A, a distal tip 70 includes a main body portion 71. The main body portion 71 is similar to the main body portion 31, except that the shapes are different. The distal tip 70 includes, between the distal end and the distal end portion 14 of the shaft 10, an enlarged diameter portion 72 where the outer diameter is largest. The enlarged diameter portion 72 has a predetermined width in the axial direction of the shaft 10. The outer diameter at the enlarged diameter portion 72 is larger than the outer diameter DS of the distal end portion 14 of the shaft 10. A protruding portion 73 extending spirally and protruding radially outward of the main body portion 71 is provided on a surface (outer peripheral surface) of the main body portion 71 of the distal tip 70. The protruding portion 73 is provided only within the range of the enlarged diameter portion 72. According to the distal tip 70, since the distal tip 70 is provided with the protruding portion 73, it is possible to improve the penetrability into a hard lesion site.

The distal tip may be formed as a distal tip 80 illustrated in FIG. 6B. The distal tip 80 includes a main body portion 81. The main body portion 81 is similar to the main body portion 31, except that the shapes are different. The distal tip 80 includes, between the distal end and the distal end portion 14 of the shaft 10, an enlarged diameter portion 82 where the outer diameter is largest. The enlarged diameter portion 82 has a predetermined width in the axial direction of the shaft 10. The outer diameter at the enlarged diameter portion 82 is larger than the outer diameter DS of the distal end portion 14 of the shaft 10. A protruding portion 83 extending spirally and protruding radially outward of the main body portion 81 is provided on a surface of the main body portion 81 of the distal tip 80. The protruding portion 83 is provided in a range from the distal end side of the enlarged diameter portion 82 to a rear end of the enlarged diameter portion 82. According to the distal tip 80, since the protruding portion 83 is provided from the distal end side of the enlarged diameter portion 82, it is possible to further improve the penetrability into a hard lesion site.

The technique disclosed herein is not limited to the above-described embodiments and the modification, and may be modified into various modes without departing from the spirit of the above-described embodiments and the modification. For example, the following modifications can be applied.

In the above-described embodiments, the shaft 10 includes the coil body 11 as an example of a reinforcing body of the shaft 10, but the present invention is not limited to this configuration. For example, a braid may be employed as the reinforcing body. If a braid is used as the reinforcing body, the braid and the distal tips 30, 40, and 50 may be joined by welding.

For example, in the above-described fourth embodiment, a slit is provided in the distal tip 60. In the above-described fifth embodiment, a protruding portion is provided for the distal tips 70 and 80. These distal tips all include a tapered shape in which the outer diameter decreases from the enlarged diameter portion toward the distal end portion 14 of the shaft 10. However, the disclosed embodiments are not limited thereto. For example, the distal tips 40 and 50 may be formed with a slit or a protruding portion.

The present invention is not limited to the configuration of the above-described embodiments, but are indicated by the claims, and are intended to include all modifications within meanings and the scope equivalent to the claims.

1. A catheter comprising:
a hollow shaft; and
a distal tip formed of metal and connected to a distal end of the hollow shaft, wherein:
an outer diameter of the distal tip in a direction perpendicular to an axial direction of the hollow shaft is largest at an enlarged diameter portion of the distal tip positioned between a distal end of the distal tip and the distal end of the hollow shaft, and
the outer diameter of the distal tip at the enlarged diameter portion is larger than an outer diameter of the distal end of the hollow shaft.

2. The catheter according to claim 1, wherein an outer peripheral surface of the distal tip in a region sandwiching the enlarged diameter portion is a curved surface having an inclination gradually changing in a longitudinal sectional view of the catheter.

3. The catheter according to claim 1, wherein an outer peripheral surface of the distal tip is outwardly convex between the enlarged diameter portion and the distal end of the hollow shaft in a longitudinal sectional view of the catheter.

4. The catheter according to claim 1, wherein an outer peripheral surface of the distal tip is radially concave between the enlarged diameter portion and the distal end of the hollow shaft in a longitudinal sectional view of the catheter.

5. The catheter according to claim 1, wherein a slit is formed in an outer peripheral surface of the distal tip at a position distal to the enlarged diameter portion.

6. The catheter according to claim 1, further comprising:
a protruding portion formed on an outer peripheral surface of the distal tip, wherein the protruding portion extends spirally in a longitudinal direction and protrudes radially outward from the outer peripheral surface.
